SEQUENCE LISTING (4 sequences)

SEQ ID NO: 1 - DNA, 25 bp, Artificial Sequence
  Synthetic sequence
ggagattggttttgacgtttatgcg

SEQ ID NO: 2 - DNA, 29 bp, Artificial Sequence
  Synthetic sequence
aagcattaaataaagcgaatacatcctta

SEQ ID NO: 3 - DNA, 19 bp, Artificial Sequence
  Synthetic sequence
tcccagcgtcgtgattagc

SEQ ID NO: 4 - DNA, 27 bp, Artificial Sequence
  Synthetic sequence
cggcataatgattaggtatacaaaaca

(19) United States
(12) Patent Application Publication
(10) Pub. No.: US 2019/0091216 A1
(43) Pub. Date: Mar. 28, 2019

(54) MACROLIDE COMPOUNDS AND THEIR USE IN LIVER STAGE MALARIA AND RELATED DISEASE

(71) Applicant: THE JOHNS HOPKINS UNIVERSITY, Park City, UT (US)

(72) Inventors: David Sullivan, Baltimore, MD (US); Nick Kaludov, Baltimore, MD (US); Yi Liu, Baltimore, MD (US); Martin Martinov, Baltimore, MD (US)

(21) Appl. No.: 15/506,445

(22) PCT Filed: Aug. 25, 2015
(86) PCT No.: PCT/US2015/046665

§ 371 Date: Feb. 24, 2017

Related U.S. Application Data
(60) Provisional application No. 62/041,380, filed on Aug. 25, 2014.

(51) Int. Cl. A61K 31/4709; A61K 45/06; A61P 33/06

(52) U.S. Cl. CPC A61K 31/4709; A61P 33/06; A61K 45/06

(57) ABSTRACT

A novel quantum-based computational process for drug discovery and design was used to identify potential novel liver-stage anti-malarial therapeutic molecules. The approach combined the latest big-data advances in high-throughput bioassay development with fundamental scientific knowledge to generate new pharmaceutical leads. Several molecules with no previous association with anti-parasitical activity were identified. These molecules and there use in prevention and/or treatment of Plasmodium infections are provided.

### REFERENCE TO RELATED APPLICATIONS

This application claims the benefit of U.S. Provisional Patent Application No. 62/041,380, filed on Aug. 25, 2014, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Aug. 25, 2014, is named P13100-01_ST25.txt and is 1,010 bytes in size.

### BACKGROUND OF THE INVENTION

Malaria, caused by the Plasmodium parasite, is a devastating disease that has plagued mankind for centuries and continues to wreak havoc across continents, with almost half of the global population at risk for the disease every year. Malaria is the leading cause of death of children under five in sub-Saharan Africa and was responsible for over one million deaths in Africa alone in 2010. Due to the large reservoir of asymptomatic cases and the spread of antimalarial drug resistance, new strategies of intervention and effective treatment are rapidly becoming more urgent to achieve disease elimination. In particular, new, economically feasible drugs that can rapidly kill the parasite are especially crucial in light of the fact that definitive drug resistance or delayed parasite clearance has been reported for all classes of antimalarials available, including artemisinin-based combined therapy (ACT).

Chemical similarity is a central principle in ligand design, and extensive chemoinformatic studies explore multiple methods based on it. However, chemical structure alone does not provide adequate description of bio-molecular interactions, which are quantum in nature. Through molecular modeling, molecules can be considered as quantum objects: quantum representation of their activity (biological, chemical or pharmacological), not the underlying structure itself, is important. The present inventors' quantum molecular representations exhibit well-defined mathematical characteristics, which afford systematic theoretical treatment and property prediction with methods that would otherwise be computationally impossible (Malar. J., 10:274 (2011); Chem. Biol. Drug Des., 80:810-820 (2012)). Specialized machine-learning algorithms with fuzzy decision-making protocols (Fuzzy Set Syst., 69:125-139 (1995)) are then applied for retrospective data analysis to identify both active compounds and the corresponding quantum features of chemical and biological interest. The modeling data consists either of high-throughput screens of structurally diverse compounds with measured activity (EC₅₀, IC₅₀, K_{d} etc.) against the target or phenotype of interest, or of co-crystal structural data of the target and a modulator. Since structurally different entities can exhibit related quantum properties, the quantum representation of biological activity allows the identification of chemically dissimilar compounds, which are similar on a quantum level and vice versa.

As malaria and related Plasmodium infections are still a global health threat, there continues to exist a need for new and improved therapeutic agents to fight these diseases.

### SUMMARY OF THE INVENTION

In accordance with one or more embodiments the present inventors used new liver-stage quantum models based on experimental phenotypic data on compounds in a liver-stage malaria bioassay for identifying novel antimalarial drugs.

In accordance with an embodiment, the present invention provides a compound selected from the group consisting of:

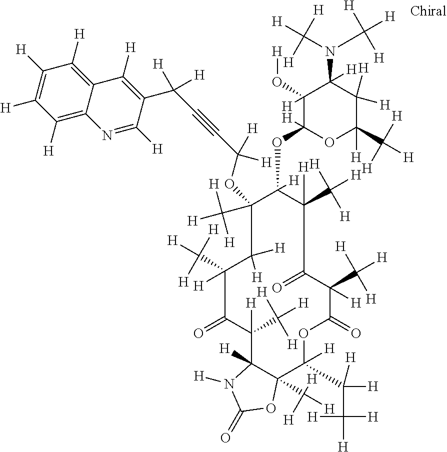 [SMILES: CC[C@H]1OC(=O)[C@H](C)C(=O)[C@H](C)[C@@H](O[C@@H]2O[C@H](C)C[C@H](N(C)C)[C@H]2O)[C@@](C)(OCC#CCc2cnc3ccccc3c2)C[C@@H](C)C(=O)[C@H](C)[C@H]2NC(=O)O[C@@]21C]

(1R,2R,4R,6S,7R,8R,10R,13R,14S)-7-[(2S,3R,4S,6R)-4-(dimethylamino)-3-hydroxy-6-methyloxan-2-yl]oxy-13-ethyl-2,4,6,8,10,14-hexamethyl-6-(4-quinolin-3-ylbut-2-ynoxy)-12,15-dioxa-17-azabicyclo[12.3.0]heptadecane-3,9,11,16-tetrone (CHEMBL440116);

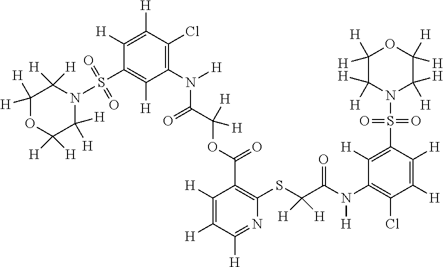 [SMILES: O=C(COC(=O)c1cccnc1SCC(=O)Nc1cc(S(=O)(=O)N2CCOCC2)ccc1Cl)Nc1cc(S(=O)(=O)N2CCOCC2)ccc1Cl]

[2-(2-chloro-5-morpholin-4-ylsulfonylanilino)-2-oxoethyl] 2-[2-(2-chloro-5-morpholin-4-ylsulfonylanilino)-2-oxoethyl]sulfanylpyridine-3-carboxylate (T0507-9950);

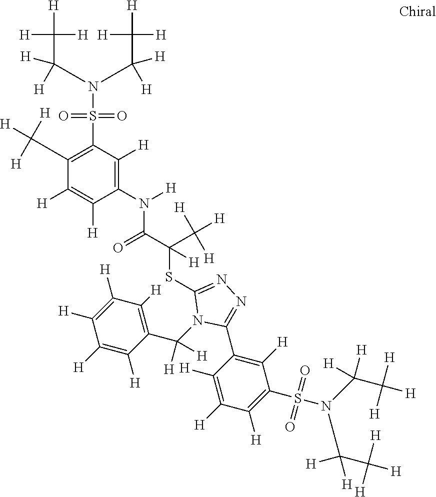 [SMILES: CCN(CC)S(=O)(=O)c1cccc(-c2nnc(SC(C)C(=O)Nc3ccc(C)c(S(=O)(=O)N(CC)CC)c3)n2Cc2ccccc2)c1]

2-[[4-benzyl-5-[3-(diethylsulfamoyl)phenyl]-1,2,4-triazol-3-yl]sulfanyl]-N-[3-(diethylsulfamoyl)-4-methylphenyl]propanamide (T5531873);

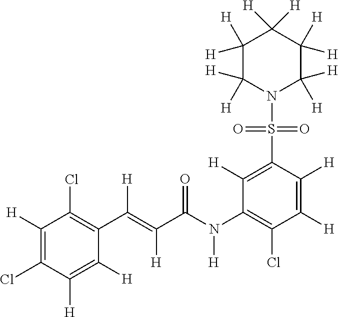 [SMILES: O=C(/C=C/c1ccc(Cl)cc1Cl)Nc1cc(S(=O)(=O)N2CCCCC2)ccc1Cl]

(E)-N-(2-chloro-5-piperidin-1-ylsulfonylphenyl)-3-(2,4-dichorophenyl)prop-2-enamide (T0510-7064); and

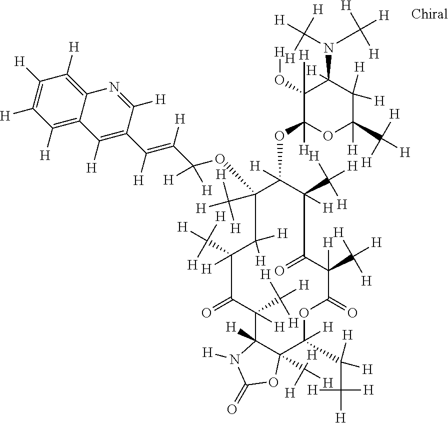 [SMILES: CC[C@H]1OC(=O)[C@H](C)C(=O)[C@H](C)[C@@H](O[C@@H]2O[C@H](C)C[C@H](N(C)C)[C@H]2O)[C@@](C)(OC/C=C/c2cnc3ccccc3c2)C[C@@H](C)C(=O)[C@H](C)[C@H]2NC(=O)O[C@@]21C]

(1R,2R,4R,6S,7R,8R,10R,13R,14S)-7-[(2S,3R,4S,6R)-4-(dimethylamino)-3-hydroxy-6-methyloxan-2-yl]oxy-13-ethyl-2,4,6,8,10,14-hexamethyl-6-[(E)-3-quinolin-3-ylprop-2-enoxy]-12,15-dioxa-17-azabicyclo[12.3.0]heptadecane-3,9,11,16-tetrone (Cethromycin); or a salt, solvate, stereoisomer, or prodrug thereof, for use in formulation of a medicament, preferably for use in the prevention or treatment of a Plasmodium infection in a subject.

In accordance with another embodiment, the present invention provides a pharmaceutical composition comprising one or more compounds, salts, solvates, stereoisomers, or prodrugs described above, and a pharmaceutically acceptable carrier.

In accordance with a further embodiment, the present invention provides a pharmaceutical composition comprising one or more compounds, salts, solvates, stereoisomers, or prodrugs described above, at least one or more additional anti-malarial compounds, and a pharmaceutically acceptable carrier.

In accordance with an embodiment, the present invention provides a pharmaceutical composition comprising primaquin, cethromycin, and a pharmaceutically acceptable carrier.

In accordance with an embodiment, the present invention provides a method of preventing or treating a Plasmodium infection in a subject comprising administering an effective amount of a compound, salt, solvate, stereoisomer, or prodrugs described above, or the pharmaceutical compositions described above.

In accordance with another embodiment, the present invention provides a method of preventing or treating a Plasmodium infection in a subject comprising administering an effective amount of a compound, salt, solvate, stereoisomer, or prodrugs described above, or the pharmaceutical compositions described above and at least one or more additional anti-malarial compounds.

### BRIEF DESCRIPTION OF THE DRAWINGS

FIGS. 1A-1D depict liver-stage quantum components. Quantum similarities of the compound GNF-Pf-1498, and the quinoline-macrolide hybrid compound cethromycin, that is related to CHEMBL440116, are shown.

FIG. 2 shows chemical structures of the compound CHEMBL440116, and other identified molecules which were acquired and tested.

FIG. 3 shows in vitro inhibition of liver stage malaria. 3A. Three of the new compounds indicated 30%, 96% and 55% inhibition while cethromycin alone at 20 μM had 54% inhibition. The individual components of cethromycin-quinoline and erythromycin, were inactive. Error is standard error of the mean of duplicate wells performed in biologic replicate. 3B. Image of near 95% inhibition by T5531873. 50,000 Hepa1-6 cells were seeded in each well 24 hours prior to infection with 50,000 P. berghei sporozoites. The 2E6 anti-HSP70 antibody was used for immunofluorescent numeration of infected cells.

FIG. 4 depicts in vivo inhibition of malarial parasites. Approximately 10,000 sporozoites were inoculated by tail vein injection and mice were sacrificed 40 hours later, livers were harvested, placed in RNAzol and parasite levels determined by realtime PCR from cDNA from reverse transcription. Relative fluorescent units were compared to control to determine percent inhibition. Cethromycin (CET) was administered only once while the other drugs were given twice 24 hours apart from each dose. Two drugs related to CET, quinolone (QN) and erythromycin (ERY), had only marginal effect on parasite growth. CET's effectiveness increased with dosage, reaching 60% reduction at 50 mg/kg. CET was also able to eliminate parasite infection when combined with low dose of PQ. All three novel compounds (T0507-9950, T5531873, T0510-7064) demonstrated significant inhibitory effect on parasite proliferation. Error is standard error of mean of three mice with real time PCR performed in duplicate for transcript levels in each mouse.

### DETAILED DESCRIPTION OF THE INVENTION

In accordance with an embodiment, the present invention provides a compound selected from the group consisting of:

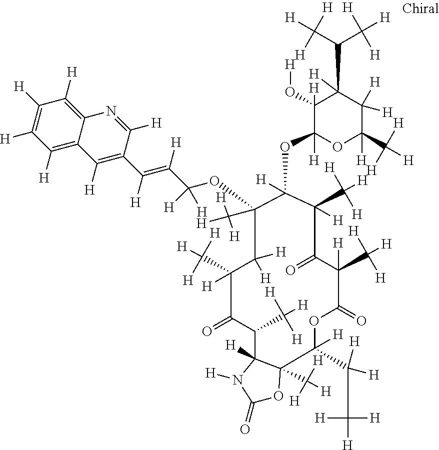 [SMILES: CC[C@H]1OC(=O)[C@H](C)C(=O)[C@H](C)[C@@H](O[C@@H]2O[C@H](C)C[C@H](C(C)C)[C@H]2O)[C@@](C)(OC/C=C/c2cnc3ccccc3c2)C[C@@H](C)C(=O)[C@H](C)[C@H]2NC(=O)O[C@@]21C]

(1R,2R,4R,6S,7R,8R,10R,13R,14S)-7-[(2S,3R,4S,6R)-4-(dimethylamino)-3-hydroxy-6-methyloxan-2-yl]oxy-13-ethyl-2,4,6,8,10,14-hexamethyl-6-(4-quinolin-3-ylbut-2-ynoxy)-12,15-dioxa-17-azabicyclo[12.3.0]heptadecane-3,9,11,16-tetrone (CHEMBL440116);

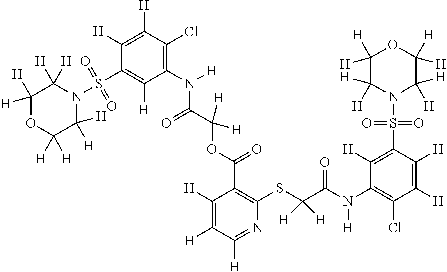 [SMILES: O=C(COC(=O)c1cccnc1SCC(=O)Nc1cc(S(=O)(=O)N2CCOCC2)ccc1Cl)Nc1cc(S(=O)(=O)N2CCOCC2)ccc1Cl]

[2-(2-chloro-5-morpholin-4-ylsulfonylanilino)-2-oxoethyl] 2-[2-(2-chloro-5-morpholin-4-ylsulfonylanilino)-2-oxoethyl]sulfanylpyridine-3-carboxylate (T0507-9950);

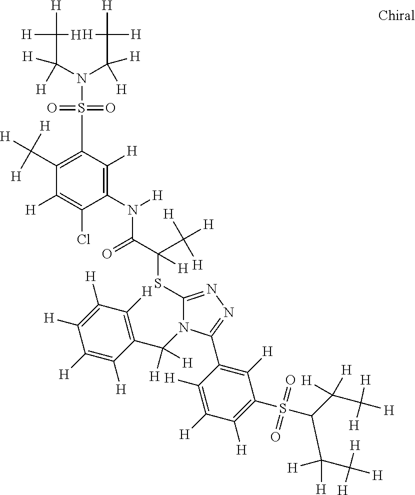 [SMILES: CCC(CC)S(=O)(=O)c1cccc(-c2nnc(SC(C)C(=O)Nc3cc(S(=O)(=O)N(CC)CC)c(C)cc3Cl)n2Cc2ccccc2)c1]

2-[[4-benzyl-5-[3-(diethylsulfamoyl)phenyl]-1,2,4-triazol-3-yl]sulfanyl]-N-[3-(diethylsulfamoyl)-4-methylphenyl]propanamide (T5531873);

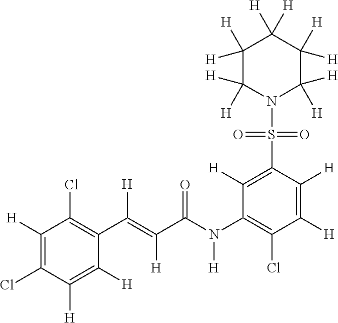 [SMILES: O=C(/C=C/c1ccc(Cl)cc1Cl)Nc1cc(S(=O)(=O)N2CCCCC2)ccc1Cl]

(E)-N-(2-chloro-5-piperidin-1-ylsulfonylphenyl)-3-(2,4-dichlorophenyl)prop-2-enamide (T0510-7064); and

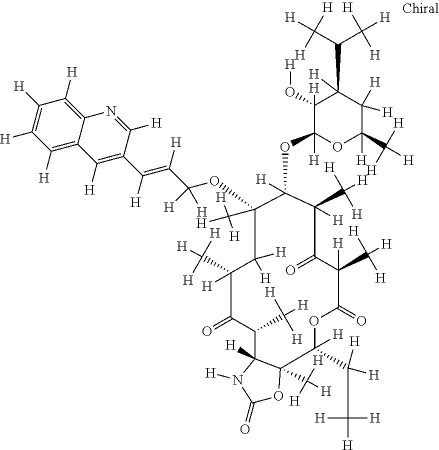 [SMILES: CC[C@H]1OC(=O)[C@H](C)C(=O)[C@H](C)[C@@H](O[C@@H]2O[C@H](C)C[C@H](C(C)C)[C@H]2O)[C@@](C)(OC/C=C/c2cnc3ccccc3c2)C[C@@H](C)C(=O)[C@H](C)[C@H]2NC(=O)O[C@@]21C]

(1R,2R,4R,6S,7R,8R,10R,13R,14S)-7-[(2S,3R,4S,6R)-4-(dimethylamino)-3-hydroxy-6-methyloxan-2-yl]oxy-13-ethyl-2,4,6,8,10,14-hexamethyl-6-[(E)-3-quinolin-3-ylprop-2-enoxy]-12,15-dioxa-17-azabicyclo[12.3.0]heptadecane-3,9,11,16-tetrone (Cethromycin); or a salt, solvate, stereoisomer, or prodrug thereof, for use in formulation of a medicament, preferably for use in the prevention or treatment of a Plasmodium infection in a subject.

Included within the compounds of the present invention are the tautomeric forms of the disclosed compounds, isomeric forms including diastereoisomers, and the pharmaceutically-acceptable salts thereof. The term “pharmaceutically acceptable salts” embraces salts commonly used to form alkali metal salts and to form addition salts of free acids or free bases. Examples of acids which may be employed to form pharmaceutically acceptable acid addition salts include such inorganic acids as hydrochloric acid, sulphuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine.

Suitable pharmaceutically acceptable salts of the compounds of the present invention include, for example, acid addition salts which may, for example, be formed by mixing a solution of the compound according to the invention with a solution of a pharmaceutically acceptable acid, such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. All of these salts may be prepared by conventional means by reacting, for example, the appropriate acid or base with the corresponding compounds of the present invention.

Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicines, the salts of the compounds of the present invention should be pharmaceutically acceptable salts. Other salts may, however, be useful in the preparation of the compounds according to the invention or of their pharmaceutically acceptable salts.

In addition, embodiments of the invention include hydrates of the compounds of the present invention. The term “hydrate” includes but is not limited to hemihydrate, monohydrate, dihydrate, trihydrate and the like. Hydrates of the compounds of the present invention may be prepared by contacting the compounds with water under suitable conditions to produce the hydrate of choice.

As defined herein, in one or more embodiments, “contacting” means that the one or more compounds of the present invention are introduced into a sample having at least one Plasmodium organism, including for example, Plasmodium falciparum, and appropriate enzymes or reagents, in a test tube, flask, tissue culture, chip, array, plate, microplate, capillary, or the like, and incubated at a temperature and time sufficient to permit binding of the at least one compounds of the present invention to interact with the organism.

In an embodiment, the pharmaceutical compositions of the present invention comprise the compounds of the present invention together with a pharmaceutically acceptable carrier.

In a further embodiment, the present invention provides a method of treating a Plasmodium infection in a subject, the method comprising administering to the subject, a pharmaceutical composition comprising at least one compound of the present invention. In another embodiment, the method comprises administering to the subject, a pharmaceutical composition comprising at least one compound of the present invention, and at least one additional compound suitable for use in treating a Plasmodium infection, with a pharmaceutically acceptable carrier, in an effective amount to inhibit, suppress or treat symptoms of the infection.

It will be understood that the term “Plasmodium infection” means an infection of the subject with Plasmodium falciparum, Plasmodium berghei, and Plasmodium vivax and related organisms.

Suitable compounds for use in treating a Plasmodium infection include, for example, the artemisinins, sulfadoxine, pyrimethamine, doxycycline, azithromycin, atovaquone, tetracycline, other antifolates like trimethoprim, sulfamethoxazole, quinolones, primaquin and clindamycin.

In accordance with an embodiment, the present invention provides the use of a compound selected from the group consisting of:

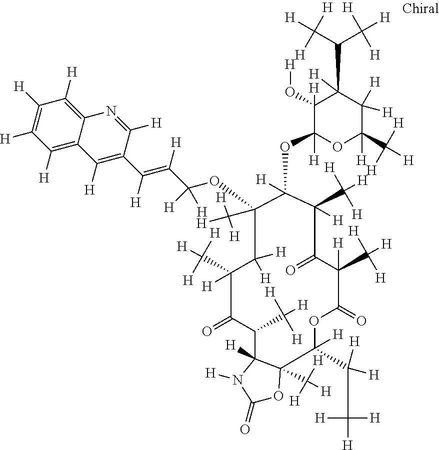 [SMILES: CC[C@H]1OC(=O)[C@H](C)C(=O)[C@H](C)[C@@H](O[C@@H]2O[C@H](C)C[C@H](C(C)C)[C@H]2O)[C@@](C)(OC/C=C/c2cnc3ccccc3c2)C[C@@H](C)C(=O)[C@H](C)[C@H]2NC(=O)O[C@@]21C]

(1R,2R,4R,6S,7R,8R,10R,13R,14S)-7-[(2S,3R,4S,6R)-4-(dimethylamino)-3-hydroxy-6-methyloxan-2-yl]oxy-13-ethyl-2,4,6,8,10,14-hexamethyl-6-(4-quinolin-3-ylbut-2-ynoxy)-12,15-dioxa-17-azabicyclo[12.3.0]heptadecane-3,9,11,16-tetrone (CHEMBL440116);

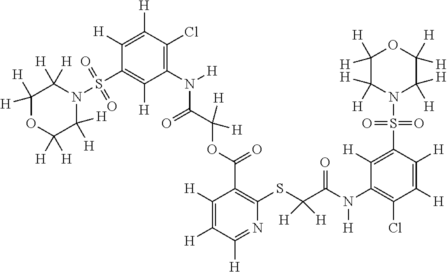 [SMILES: O=C(COC(=O)c1cccnc1SCC(=O)Nc1cc(S(=O)(=O)N2CCOCC2)ccc1Cl)Nc1cc(S(=O)(=O)N2CCOCC2)ccc1Cl]

[2-(2-chloro-5-morpholin-4-ylsulfonylanilino)-2-oxoethyl] 2-[2-(2-chloro-5-morpholin-4-ylsulfonylanilino)-2-oxoethyl]sulfanylpyridine-3-carboxylate (T0507-9950);

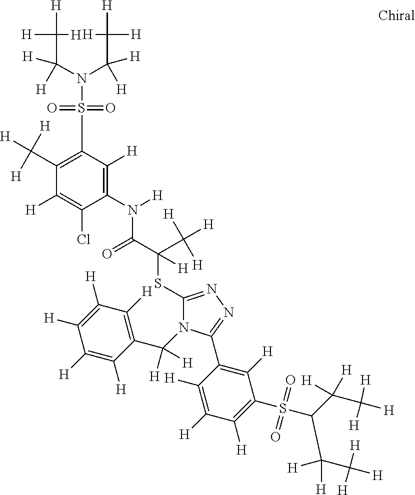 [SMILES: CCC(CC)S(=O)(=O)c1cccc(-c2nnc(SC(C)C(=O)Nc3cc(S(=O)(=O)N(CC)CC)c(C)cc3Cl)n2Cc2ccccc2)c1]

2-[[4-benzyl-5-[3-(diethylsulfamoyl)phenyl]-1,2,4-triazol-3-yl]sulfanyl]-N-[3-(diethylsulfamoyl)-4-methylphenyl]propanamide (T5531873);

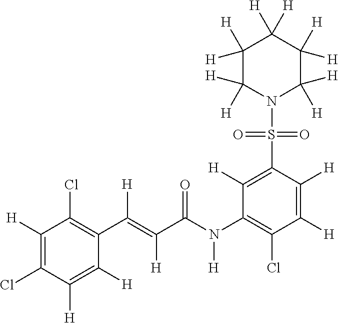 [SMILES: O=C(/C=C/c1ccc(Cl)cc1Cl)Nc1cc(S(=O)(=O)N2CCCCC2)ccc1Cl]

(E)-N-(2-chloro-5-piperidin-1-ylsulfonylphenyl)-3-(2,4-dichlorophenyl)prop-2-enamide (T0510-7064); and

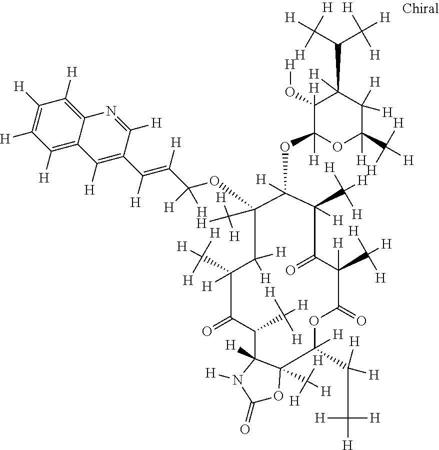 [SMILES: CC[C@H]1OC(=O)[C@H](C)C(=O)[C@H](C)[C@@H](O[C@@H]2O[C@H](C)C[C@H](C(C)C)[C@H]2O)[C@@](C)(OC/C=C/c2cnc3ccccc3c2)C[C@@H](C)C(=O)[C@H](C)[C@H]2NC(=O)O[C@@]21C]

(1R,2R,4R,6S,7R,8R,10R,13R,14S)-7-[(2S,3R,4S,6R)-4-(dimethylamino)-3-hydroxy-6-methyloxan-2-yl]oxy-13-ethyl-2,4,6,8,10,14-hexamethyl-6-[(E)-3-quinolin-3-ylprop-2-enoxy]-12,15-dioxa-17-azabicyclo[12.3.0]heptadecane-3,9,11,16-tetrone (Cethromycin); or a salt, solvate, stereoisomer, or prodrug thereof, and a pharmaceutically acceptable carrier, in an effective amount, to prevent and/or treat a plasmodium infection in a subject. The use can include additional antimalarial compounds in addition.

Embodiments of the invention include a process for preparing pharmaceutical products comprising the compounds, salts, solvates or stereoisomers thereof. The term “pharmaceutical product” means a composition suitable for pharmaceutical use (pharmaceutical composition), as defined herein. Pharmaceutical compositions formulated for particular applications comprising the Plasmodium inhibitors of the present invention are also part of this invention, and are to be considered an embodiment thereof.

As used herein, the term “treat,” as well as words stemming therefrom, includes preventative as well as disorder remitative treatment. The terms “reduce”, “suppress” and “inhibit,” as well as words stemming therefrom, have their commonly understood meaning of lessening or decreasing. These words do not necessarily imply 100% or complete treatment, reduction, suppression, or inhibition.

With respect to pharmaceutical compositions described herein, the pharmaceutically acceptable carrier can be any of those conventionally used, and is limited only by physico-chemical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s), and one which has little or no detrimental side effects or toxicity under the conditions of use. Examples of the pharmaceutically acceptable carriers include soluble carriers such as known buffers which can be physiologically acceptable (e.g., phosphate buffer) as well as solid compositions such as solid-state carriers or latex beads.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Intravenous vehicles include, for example, fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

In addition, in an embodiment, the compounds of the present invention may further comprise, for example, binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzlkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular compound, as well as by the particular method used to administer the compound. Accordingly, there are a variety of suitable formulations of the pharmaceutical composition of the invention. The following formulations for parenteral, subcutaneous, intravenous, intramuscular, intraarterial, intrathecal and interperitoneal administration are exemplary, and are in no way limiting. More than one route can be used to administer the compounds of the present invention, and in certain instances, a particular route can provide a more immediate and more effective response than another route.

Suitable soaps for use in parenteral formulations include, for example, fatty alkali metal, ammonium, and triethanolamine salts, and suitable detergents include, for example, (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the compound of the present invention or a salt, solvate or stereoisomer thereof, in solution. Preservatives and buffers may be used. In order to minimize or eliminate irritation at the site of injection, such compositions may contain one or more nonionic surfactants, for example, having a hydrophile-lipophile balance (HLB) of from about 12 to about 17. The quantity of surfactant in such formulations will typically range from about 5% to about 15% by weight. Suitable surfactants include, for example, polyethylene glycol sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

For purposes of the invention, the amount or dose of the compound of the present invention, or a salt, solvate or stereoisomer thereof, administered should be sufficient to effect, e.g., a therapeutic or prophylactic response, in the subject over a reasonable time frame. The dose will be determined by the efficacy of the particular compound and the condition of a human, as well as the body weight of a human to be treated.

The dose of the compound of the present invention, or a salt, solvate or stereoisomer thereof, also will be determined by the existence, nature and extent of any adverse side effects that might accompany the administration of a particular compound. Typically, an attending physician will decide the dosage of the compound with which to treat each individual patient, taking into consideration a variety of factors, such as age, body weight, general health, diet, sex, compound to be administered, route of administration, and the severity of the condition being treated. By way of example, and not intending to limit the invention, the dose of the compound can be about 0.001 to about 100 mg/kg body weight of the subject being treated/day.

Alternatively, the compound of the present invention, or a salt, solvate or stereoisomer thereof, can be modified into a depot form, such that the manner in which the compound is released into the body to which it is administered is controlled with respect to time and location within the body (see, for example, U.S. Pat. No. 4,450,150). Depot forms of compound can be, for example, an implantable composition comprising the compound and a porous or non-porous material, such as a polymer, wherein compound is encapsulated by or diffused throughout the material and/or degradation of the non-porous material. The depot is then implanted into the desired location within the body and the compounds are released from the implant at a predetermined rate.

In one embodiment, the compounds of the present invention, or salts, solvates or stereoisomers thereof, provided herein can be controlled release compositions, i.e., compositions in which the one or more compounds are released over a period of time after administration. Controlled or sustained release compositions include formulation in lipophilic depots (e.g., fatty acids, waxes, oils). In another embodiment the composition is an immediate release composition, i.e., a composition in which all or substantially all of the RNase H inhibitor is released immediately after administration.

In yet another embodiment, the compounds of the present invention can be delivered in a controlled release system. For example, the agent may be administered using intravenous infusion, an implantable osmotic pump, a transdermal patch, or other modes of administration. In an embodiment, a pump may be used. In one embodiment, polymeric materials can be used. In yet another embodiment, a controlled release system can be placed in proximity to the therapeutic target, i.e., the brain, thus requiring only a fraction of the systemic dose (see, e.g., Design of Controlled Release Drug Delivery Systems, Xiaoling Li and Bhaskara R. Jasti eds. (McGraw-Hill, 2006)).

The compounds of the present invention, or salts, solvates or stereoisomers thereof, may also include incorporation of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In accordance with the present invention, the compounds may be modified by, for example, the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection, than do the corresponding unmodified compounds. Such modifications may also increase the compounds' solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently, or in lower doses than with the unmodified compound.

Using the quantum similarity modeling approach, the present inventors discovered a drug, ready to be evaluated in humans for malaria elimination and identified several additional orally bioavailable lead molecules that inhibited the parasite in an animal liver-stage model. The research was performed in less than a year, at a fraction of the cost of similar drug discovery efforts. Equally importantly, however, the model-discovered liver-stage quantum properties have been experimentally validated. The existing commercial compounds T0507-9950, T5531873 and T0510-7064 could serve as excellent leads in a traditional medicinal chemistry optimization. Furthermore, since this type of molecular modeling does not depend on chemical structures but on abstract quantum components that can be found on multiple chemically different compounds, quantum similarity allows the design of novel chemical entities with simultaneously optimized target activity, therapeutic efficacy and favorable pharmacological characteristics. Once a quantum model has been validated, it provides a powerful tool for de novo lead discovery by inverse molecular construction, which combines on the same chemical structure the identified quantum molecular attributes responsible for liver-stage antimalarial activity and other physicochemical properties of interest.

One of the newly identified compounds, Cethromycin [ABT 773], a macrolide-quinoline hybrid, is a drug with extensive safety profile that was active with more than a log decrease in mice synergizing with primaquine. Cethromycin is a erythromycin and quinoline nucleus hybrid. Individually erythromycin and quinoline have no activity in vitro or in the mouse model, but cethromycin was active. Cethromycin has been used safely in over 5,000 humans in efficacy studies for single day dosing for bacterial pneumonia. The safety and pharmacokinetics are a suitable match for a potential safe effective human liver stage malaria drug.

### EXAMPLES

Quantum Comparison Methods

Structure representation—localized electron-density descriptors for molecular modeling: Well-defined chemical subsystems, together with their associated local, spatially-resolved properties, are very useful in drug discovery (Zartler, E., and M. Shapiro. 2008. Fragment-Based Drug Discovery. A Practical Approach. John Wiley & Sons). On a theoretical level, these properties serve as powerful descriptors for molecular modeling and design. Notions from Density Functional Theory and Topological Theory of Atoms in Molecules can be combined to rigorously define and compute a complete set of such localized, electron-density descriptors.

In general, Non-Relativistic Quantum Mechanics (QM) provides the proper level of physical theory for treatment of molecular and bio-molecular systems. However, many intuitive chemical concepts are not directly related to the corresponding wave function, a state-vector in Hilbert space, which is difficult to partition into chemically meaningful subsystems (J Chem Phys 100:2900-2909 (1994)).

Density Functional Theory (DFT) provides a systematic framework for inferring chemistry-related information from QM calculations. This is achieved through the use of the electron density, ρ(r), a real, nonnegative Cartesian function connected to the N-electron molecular wave function ψ by

ρ(r)=∫|ψ(x,x₁, . . . ,x_{N-1}|²dsdx₁. . . dx_{N-1},

where x={s, r} is the four-dimensional spin-spatial coordinate. As the famous Hohenberg-Kohn theorem shows, ρ(r) determines all ground-state properties of the entire system, including its chemical and biochemical features.

Furthermore, the Topological Theory of Atoms in Molecules (AIM) (Matta, C., and R. Boyd. 2007. The Quantum Theory of Atoms in Molecules. Wiley-VCH) uses ρ(r) to partition molecules into precise atomic subsystems. These atomic subsystems are bounded by zero-flux surfaces S, which obey the equation

∇r∈S n(r)·∇ρ(r)=0,

where n(r) is the vector normal to S at r and ρ(r) is the corresponding electron density.

It is natural to combine DFT and AIM, together with their respective computational algorithms, in a single formalism for studying local molecular properties from first principles. This formalism has yielded meaningful interpretations of many general chemical concepts, such as energy partitioning, atomic softness, electronegativity equalization, atomic reactivity indices, etc. Augmented with the electrostatic potential, this electron density-based methodology has been applied to quantitative structure-activity relationship studies. It also produced the molecular descriptors employed in the modeling effort described here. Importantly, when applied as descriptors, these electron-density transforms define a proper metric (molecular similarity measure) in the modeling space, and allow the use of rigorous mathematical techniques.

Modelling architecture—fuzzy decision networks: Molecular modeling is a multi-step process:

{Sᵢ,Pᵢ}→{D_{i,j}(S),Pᵢ}→P[D(S)].

The starting point, {Sᵢ, Pᵢ}, called a training set, is a set of molecular structures Sᵢ for which a particular property of interest P has been measured. In the first step, descriptor calculation, every structure is reduced to some form, typically a list of real numbers {Dⱼ}, which can be modeled statistically. The second step, actual modeling, attempts to find a model—a general mapping between property P and structure S through descriptors D. If successful, the model would have predictive power that can be applied to structures for which no measurement exists. Naturally, the predictive power of the model depends on the quality (accuracy, diversity, etc.) of the training set as well as descriptor properties and modeling architecture.

Both powerful descriptors and proper modeling architecture are crucial for successful molecular modeling and compound discovery. Ideally, the modeling architecture should be chosen in accordance with the underlying fundamental processes of the system, and not with the type of available numerical data. Complex biochemical interactions involve local attributes of distinct and diverse molecular structures, which are best modeled with discrete combinatorial methods rather than continuous multivariate techniques. Still, inherent weaknesses of traditional molecular descriptors require the use of such continuous multivariate techniques. As sophisticated as some of these techniques are, they cannot always compensate for the shortcomings of the underlying molecular-structure representations.

A straightforward machine-learning algorithm using fuzzy-logic decisions easily discovers the relationship between quantum components and specific interaction patterns. In its simplest implementation, the modeling algorithm produces a model in the form of a fuzzy decision tree. Each tree node corresponds to a single descriptor (interaction constraint). In a fully resolved decision tree, terminal nodes contain only either active or inactive molecules. Furthermore, each terminal node is fully characterized statistically—if a molecule belongs to it, the prediction is qualified by associated confidence intervals and other statistical parameters. A model in the form of a decision tree is easy to interpret. Each tree path that contains an active terminal node also contains a set of nodes (quantum components) that define the interaction pattern common to all training-set molecules belonging to this terminal. The fuzzy decision tree formalism can be generalized to more powerful fuzzy decision algorithms. Given a diverse training set of structures with known inhibition, the modeling effort produces a decision network characterizing all present interaction patterns in terms of activity-controlling descriptors, which can be visualized.

Synthesis of Cethromycin and Quality Testing.

Cethromycin was prepared according to literature procedure starting from commercially available erythromycin (Tetrahedron 60:10171-10180 (2010)). The final compound was purified directly on silica gel using a Biotage Isolera One automated purification unit (0-16% methanol in dichloromethane over 20 column volumes). The isolated compound was concentrated under reduced pressure and placed under high vacuum for two days: 15.0 mg, 63% yield (final step); 1H NMR (400 MHz, CDCl3) δ 9.03 (s, 1H), 8.18 (s, 1H), 8.06 (d, J=8.4 Hz, 1H), 7.83 (d, J=8.1 Hz, 1H), 7.64 (t, J=7.6 Hz, 1H), 7.51 (t, J=7.5 Hz, 1H), 6.56 (d, J=16.0 Hz, 1H), 6.18 (dt, J=15.7, 6.8 Hz, 1H), 5.55 (s, 1H), 4.93 (dd, J=9.3, 3.1 Hz, 1H), 4.40 (d, J=4.2 Hz, 1H), 4.38 (d, J=6.4 Hz, 1H), 3.96 (q, J=6.7 Hz, 1H), 3.90 (s, 1H), 3.84 (dd, J=11.8, 6.6 Hz, 1H), 3.70 (dd, J=11.9, 7.1 Hz, 1H), 3.62-3.51 (m, 1H), 3.33-3.22 (m, 1H), 3.23-3.14 (m, 1H), 2.96 (q, J=6.5 Hz, 1H), 2.76-2.67 (m, 1H), 2.67-2.58 (m, 1H), 2.40 (s, 6H), 2.07 (bs, 1H), 1.92-1.85 (m, 1H), 1.83 (dd, J=12.7, 5.5 Hz, 1H), 1.73 (d, J=12.4 Hz, 2H), 1.67 (d, J=13.8 Hz, 1H), 1.57-1.51 (m, 1H), 1.49 (s, 3H), 1.42 (s, 3H), 1.40 (d, J=8.0 Hz, 3H), 1.38 (d, J=6.6 Hz, 3H), 1.33-1.22 (m, 1H), 1.19 (d, J=6.0 Hz, 3H), 1.14 (d, J=7.8 Hz, 3H), 1.11 (d, J=6.3 Hz, 3H), 0.79 (t, J=7.4 Hz, 3H); 13C NMR (101 MHz, CDCl3) δ 217.39, 205.40, 169.67, 157.77, 149.59, 147.46, 132.66, 129.88, 129.17, 129.01, 128.58, 128.07, 126.81, 102.52, 83.55, 78.71, 77.52, 76.42, 69.99, 68.95, 66.05, 64.30, 58.22, 50.85, 46.22, 45.07, 39.99, 39.04, 37.34, 29.05, 27.90, 22.62, 21.09, 20.19, 18.08, 14.47, 14.11, 13.67, 10.66; LCMS—retention time=3.747 min (4% to 100% Acetonitrile (0.05% TFA) over 7 minutes; Luna C18 column, 3 micron 3×75 mm), MS (ESI) m/z (M+H)+=765.7; HRMS (ESI) for C42H61N3O10 m/z [(M+2H)/2]+ calculated=383.7186, found=383.7183.

In Vitro Malaria Liver Stage Assay.

Drugs were purchased from either Sigma or Ambinter. In each chamber of 8-well LabTek tissue culture slides, 50,000 mouse hepatoma cells, Hepa1-6, were seeded one day before infection. Cells were normally cultured in DMEM supplemented with 10% FBS, 1×L-glutamine and 1×Pen-Strep at 37° C. and 5% CO₂. Once incubated with P. berghei sporozoites, the culture medium supplement was changed to 2.5% FBS, 1×L-glutamine and 2×Pen-Strep. Before treatment with drugs, the cells were washed four times with DMEM containing 10×Pen-Strep and 5 ug/mL fungicide. After 24 hours, fresh culture medium containing drugs was added to each chamber. Approximately 42 hours post-infection, the growth medium was removed and 100% cold methanol was added for 15 minutes to fix the cells. After washing with sterile PBS, cells were incubated in PBS with 5% FBS for blocking. Then 2E6 anti-HSP70 antibody was diluted to 10 μg/mL in PBS with 5% FBS and added in a 200 μL volume to cells. One hour later, cells were washed with PBS and incubated with 10 μg/mL Alexafour 594 anti-mouse secondary antibody. In order to visualize hepatocytes, nuclear counterstain DAPI was incubated with cells for 5 minutes and then washed off. The plastic chambers and silicone gasket were removed and slide was fully dried before mounted and sealed with cover slides. When preparation was complete, slide was taken to the fluorescence microscope for examination. Representative areas were randomly selected from each chamber and the numbers of infected cells were counted for a similar number of microscopic fields. The level of inhibition was determined by comparing the average of each chamber containing treated cells with that of the controls.

In Vivo Liver Stage Assay.

Mice were kept in Johns Hopkins Bloomberg School of Public Health mouse facility according to the ACUC animal protocol number MO09H401. Six week old C57BL/6 mice weighing about 20-22 g were divided into groups of three. 12,000 P. berghei sporozoites in a 200 μL volume were injected into mice through tail veins. Two hours after injection, different dilutions of drugs in 100-200 μL volumes were delivered to mice by oral gavage using a plastic feeding tube. A second dose of drugs was given 24 hours after the first one to the mice that were on a daily dosing regimen. Approximately 40 hours post-infection, mice infected with P. berghei sporozoites were anesthetized by inhaling Metofane® and sacrificed for harvesting whole livers. Each mouse liver was immediately put into 10 mL of Trizol® Reagent and fully homogenized. After RNA isolation, the RNA was diluted to 100 ng/mL for reverse-transcription reactions. For a 30 μL reaction, the components were set up as: 3.5 μL of nuclease-free water, 3 μL of 10× Buffer II and 10 mM dNTPs, 6 μL of MgCl₂ solution, 1.5 μL of 50 μM Random Hexamers, RNase Inhibitor and MuLV Reverse Transcriptase, 10 μL of RNA sample. cDNA products were stored at −20° C. For a 10 μL real-time quantitative PCR, the components were set up as: 0.2 μL of 10 μM forward and reverse primer 18s P. berghei, 5 μL of 2×SYBR Green PCR Master Mix, 1.6 μL of nuclease-free water, and 3 μL of cDNA sample. All test subjects were done in duplicates including positive and negative controls. When real-time PCR was finished, all data were baselined and normalized to the housekeeping gene before further analysis. The specific sequences of primers (The Core DNA Analysis Facility—JHU, Baltimore, Md.) were: 5′-GGAGATTGGTTTTGACGTTTATGCG-3′ (SEQ ID NO: 1) and 5′-AAGCATTAAATAAAGCGAATACATCCTTA-3′(SEQ ID NO: 2) for P. berghei ANKA 18s and 5′-TCCCAGCGTCGTGATTAGC-3′ (SEQ ID NO: 3) and 5′-CGGCATAATGATTAGGTATACAAAACA-3′ (SEQ ID NO: 4) for mouse HPRT.

### Example 1

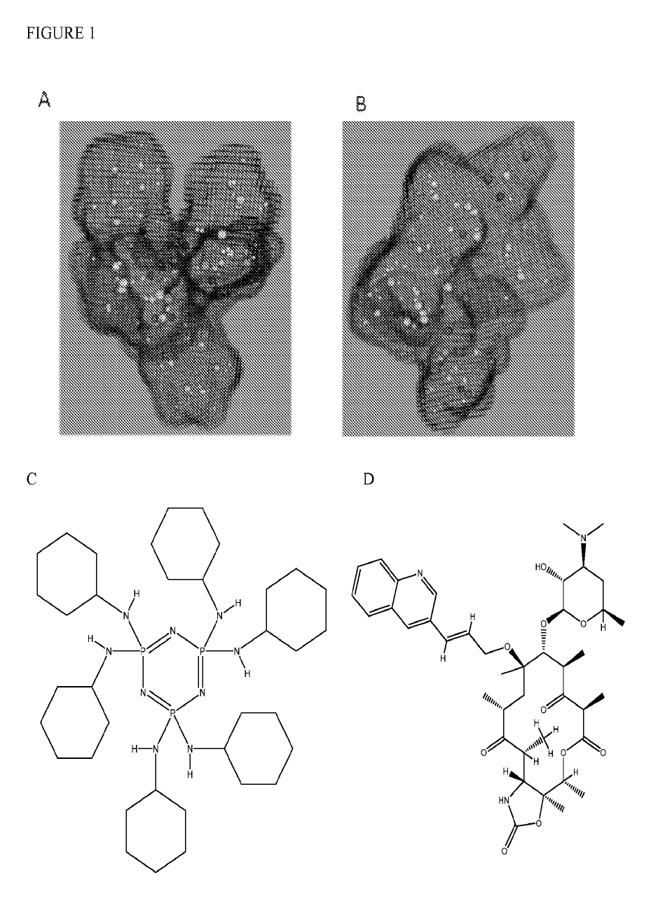

A training dataset of 5757 compounds was generated by combining data from Novartis ChEMBL-NTD HTS and additional validated liver stage antimalarial drugs from recent publications that investigated in vitro hepatocyte Plasmodium inhibition (J Infect Dis 205:1278-1286 (2012); Proc Natl Acad Sci USA 109:8511-8516 (2012); Antimicrob Agents Chemother 52:1215-1220 (2008)). The dataset was utilized to establish the quantum components (QCs) related to liver-stage inhibition. These liver-stage QCs were used as filters to virtually screen a database of 65 million commercially available compounds, which were already pre-computed in a quantum format suitable for fast processing. This in silico screen was followed by a chemical-diversity filter to assure that the identified compounds are novel and chemically different from the training set. Since QCs related to biological or other activity can be carried by structurally different compounds, the procedure calculated the quantum similarity between the QCs established from the liver-stage training set modeling and the QCs of the commercially available compounds in the database. This process identified and rank-ordered 35 different, chemically dissimilar structures from the training set, predicted by the models to be active based on their similar liver-stage activity QCs. A number of commonly accepted theoretical measures of chemical similarity were considered to estimate the novelty of the proposed compounds. These include Tanimoto coefficients based on pharmacological functional groups or compound fragments, as well as chemical diversity measures derived from electron density considerations. Once computed, these indices were used to create point-to-set distance metrics, which determine the dissimilarity of the considered structure from the known liver-stage active molecules (FIG. 1).

The model matches the input molecules with identified candidates over numerous quantum scoring criteria. A representative image of matching the quantum components is shown in FIG. 1 for cethromycin and GNF-Pf-1498, where the greater weight is given to the non-macrolide quinoline on cethromycin and the nitrogen-rich aromatic of GNF-Pf-1498 shown in red. Table 1 identifies the matched quantum score with the reference input molecules from the training set. Reference molecules Cyclosporin A and monensin have a liver stage inhibition of 1.7 nM and 0.001 nM respectively.

TABLE 1
Matching quantum scores of input and output molecules

| | | | | | | | | | | | |
|---|---|---|---|---|---|---|---|---|---|---|---|
| Input | CyclosporinA | 0 | 0 | 0 | 1(0; 1)(1) | 1(1; 1)(1) | 0 | 1(1; 1)(3) | 1(1; 1)(4) | 0 | 1(1; 2)(1; 2) |
| output | T5531873 | 1(1; 1)(1) | 1(1; 1)(1) | 1(0; 1)(1) | 1(0; 1)(1) | 1(0; 1)(1) | 0 | 1(1; 1)(3) | 1(0; 5)(1; 2; 3; | 0 | 1(0; 2)(1; 2) |
| | | | | | | | | | 4; 5) | | |
| output | T0510-7064 | 1(1; 1)(1) | 1(1; 1)(1) | 1(0; 1)(1) | 1(0; 1)(1) | 1(0; 1)(1) | 0 | 1(1; 1)(3) | 1(0; 5)(1; 2; 3; | 0 | 1(0; 2)(1; 2) |
| | | | | | | | | | 4; 5) | | |
| input | Monensin | 0 | 0 | 0 | 1(0; 1)(1) | 1(1; 1)(1) | 0 | 1(1; 1)(1) | 1(1; 1)(1) | 0 | 1(1; 2)(1; 2) |
| input | Telithromycin | 0 | 0 | 0 | 1(0; 1)(1) | 1(1; 1)(1) | 1(1; 1)(1) | 1(1; 1)(1) | 1(1; 1)(1) | 0 | 1(1; 2)(1; 2) |
| output | CHEMBL440116 | 0 | 0 | 0 | 1(0; 1)(1) | 1(1; 1)(1) | 1(1; 1)(1) | 1(1; 1)(1) | 1(1; 1)(1) | 0 | 1(1; 2)(1; 2) |

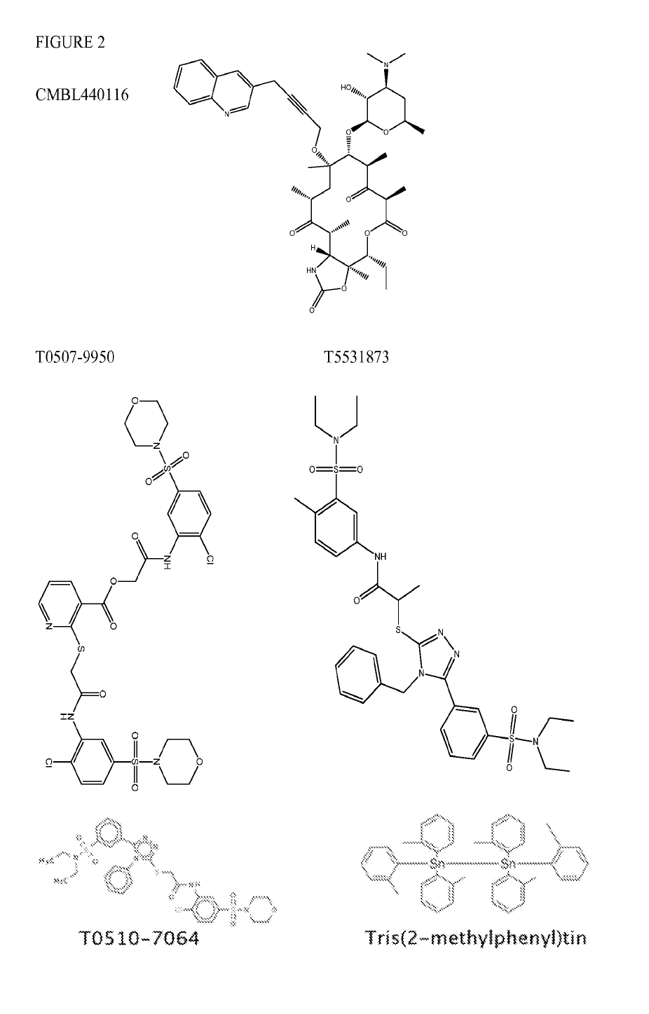

Cethromycin is chemically similar to one of the top potential candidates, bearing an allylic linker between the macrolide and the quinoline (versus a four-carbon linker containing a triple bond). The structures of the obtained compounds are shown in FIG. 2, and quantum scores and identification are in Table 2.

TABLE 2
Summary of quantum scores with molecule identifications
and reference molecules for scoring

| | Quantum | Reference | |
|---|---|---|---|
| Name | pattern score | molecule | CID |
| T0507-9950 | 19 | Cyclosporin A | 5109719 |
| T5531873 | 21 | Cyclosporin A | 16290646 |
| T0510-7064 | 20 | None | 5011386 |
| Tris(2-methylphenyl)tin | 18 | Everolimus | 50932585 |
| CHEMBL440116 | 15 | GNF-Pf-1498; | 44296067 |
| | | Monensin | |
| Cethromycin | | GNF-Pf-1498; | 447451 |
| | | Monensin | |

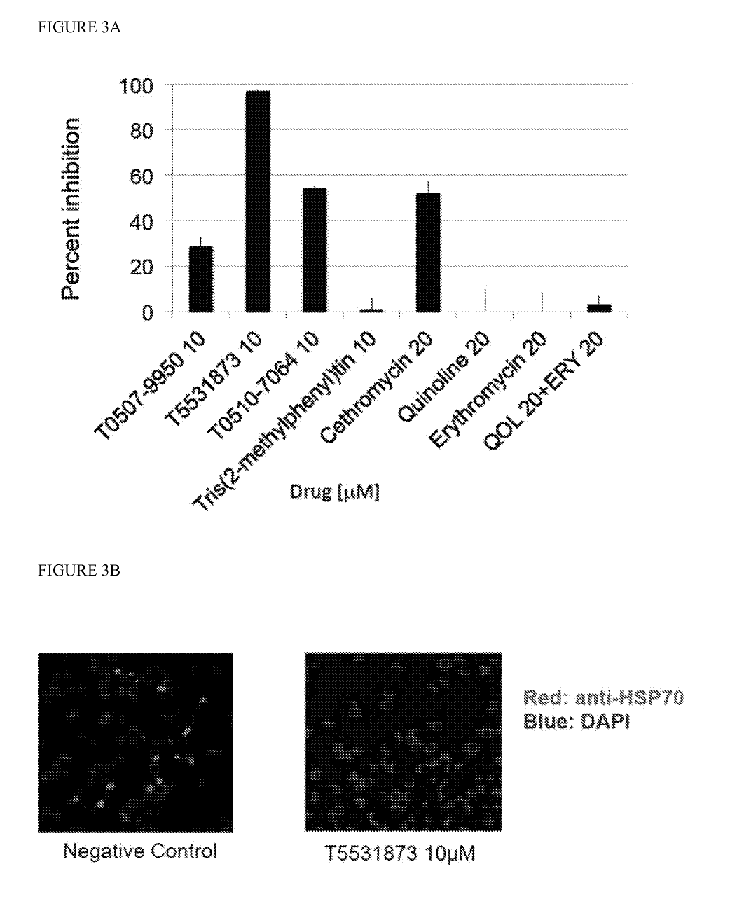

Five compounds were procured while cethromycin was synthesized. Cethromycin was directly synthesized from erythromycin by substituting the cladinose sugar at C3 with a keto-group, attaching a cyclic carbamate group at C11-C12, and tethering the quinoline moiety to the C6 alcohol. We used the in vitro P. berghei ANKA model on mouse hepatoma cells, Hepa1-6. Among all drugs and compounds tested, T5531873 at 10 μM was able to reduce parasite multiplication by over 95%, while cethromycin at 20 μM and T0510-7064 at 10 μM reduced growth by 52% and 54%, respectively (FIG. 3A). Quinoline, erythromycin and their combination were also tested to investigate the role the quinoline plays in the antimalarial activity of cethromycin. Neither quinoline, nor erythromycin, nor their combination showed any parasite inhibition in vitro. Of the five compounds tested in vitro, those that demonstrated measurable inhibitory effect on parasite infection of hepatoma cells (T0507-9950, T5531873, T0510-7064 and cethromycin) were further validated in a mouse model at a dose of 50 mg/kg. Due to limited synthesized amounts of cethromycin, only one dose was given to mice while other drugs and compounds were administered twice.

### Example 2

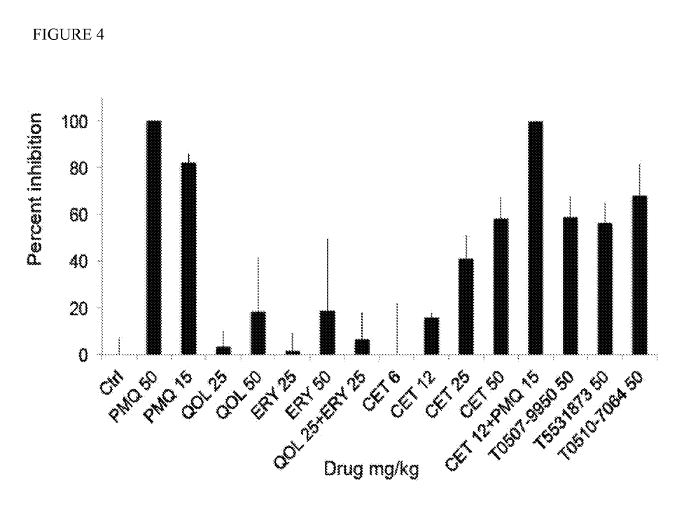

Consistent with results from the in vitro assay, quinoline, erythromycin and their combination measured only marginal inhibitory effect on parasite growth shown in FIG. 4. Although cethromycin at 12 mg/kg was only partially effective, a gradual dose response was observed after increasing the dose to 50 mg/kg, at which cethromycin reduced parasite load by 60 percent. We were able to achieve parasite elimination by combining cethromycin at a dose of 12 mg/kg with primaquine at a dose of 15 mg/kg. While cethromycin at 12 mg/kg showed minimal inhibition at about 16%, combination with low dose primaquine at 15 mg/kg (itself achieving 83% inhibition) showed greater than 99% inhibition. Based on these results, it is safe to say that cethromycin is the most potent compound tested in this study. Although the T5531873 compound was not as effective as in the in vitro assay, all three commercial compounds (T0507-9950, T5531873 and T0510-7064) reduced parasite load by more than 50 percent without causing any notable side effects in the mice after two doses. It is expected that these compounds, when combined with primaquine, will show at least effects similar to the combination of primaquine with cethromycin.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for prevention or treatment of a Plasmodium infection in a subject in need thereof, comprising administering to the subject, an effective amount of a pharmaceutical composition comprising one or more compounds selected from the group consisting of: 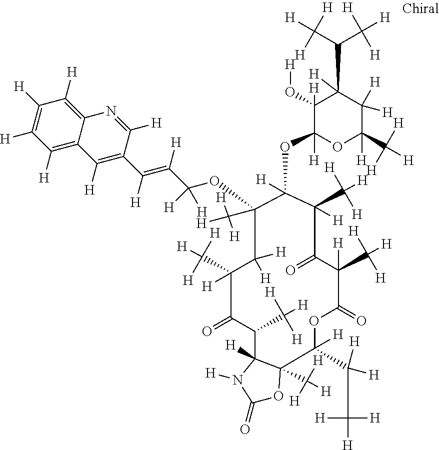 [SMILES: CC[C@H]1OC(=O)[C@H](C)C(=O)[C@H](C)[C@@H](O[C@@H]2O[C@H](C)C[C@H](C(C)C)[C@H]2O)[C@@](C)(OC/C=C/c2cnc3ccccc3c2)C[C@@H](C)C(=O)[C@H](C)[C@H]2NC(=O)O[C@@]21C]
(1R,2R,4R,6S,7R,8R,10R,13R,14S)-7-[(2S,3R,4S,6R)-4-(dimethylamino)-3-hydroxy-6-methyloxan-2-yl]oxy-13-ethyl-2,4,6,8,10,14-hexamethyl-6-(4-quinolin-3-ylbut-2-ynoxy)-12,15-dioxa-17-azabicyclo[12.3.0]heptadecane-3,9,11,16-tetrone (CHEMBL440116); 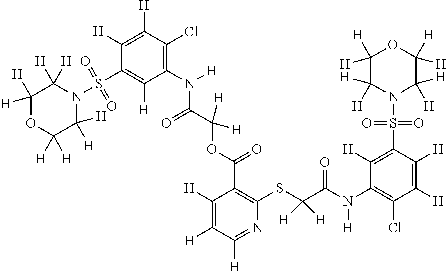 [SMILES: O=C(COC(=O)c1cccnc1SCC(=O)Nc1cc(S(=O)(=O)N2CCOCC2)ccc1Cl)Nc1cc(S(=O)(=O)N2CCOCC2)ccc1Cl]
[2-(2-chloro-5-morpholin-4-ylsulfonylanilino)-2-oxoethyl] 2-[2-(2-chloro-5-morpholin-4-ylsulfonylanilino)-2-oxoethyl]sulfanylpyridine-3-carboxylate (T0507-9950); 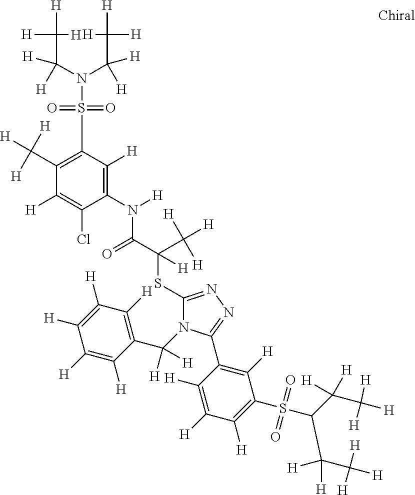 [SMILES: CCC(CC)S(=O)(=O)c1cccc(-c2nnc(SC(C)C(=O)Nc3cc(S(=O)(=O)N(CC)CC)c(C)cc3Cl)n2Cc2ccccc2)c1]
2-[[4-benzyl-5-[3-(diethylsulfamoyl)phenyl]-1,2,4-triazol-3-yl]sulfanyl]-N-[3-(diethylsulfamoyl)-4-methylphenyl]propanamide (T5531873); 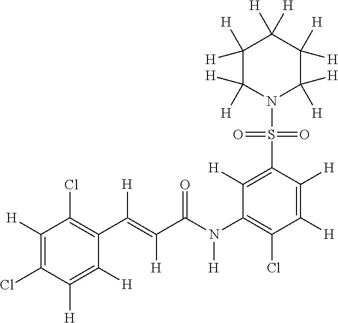 [SMILES: O=C(/C=C/c1ccc(Cl)cc1Cl)Nc1cc(S(=O)(=O)N2CCCCC2)ccc1Cl]
(E)-N-(2-chloro-5-piperidin-1-ylsulfonylphenyl)-3-(2,4-dichlorophenyl)prop-2-enamide (T0510-7064); and 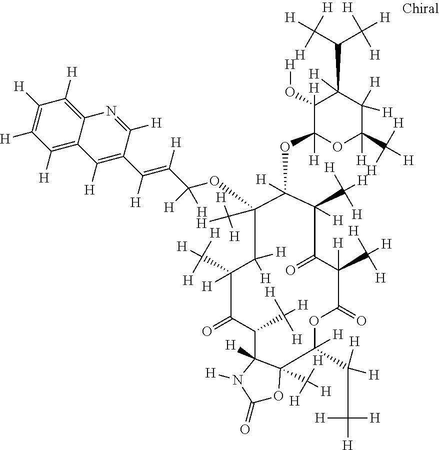 [SMILES: CC[C@H]1OC(=O)[C@H](C)C(=O)[C@H](C)[C@@H](O[C@@H]2O[C@H](C)C[C@H](C(C)C)[C@H]2O)[C@@](C)(OC/C=C/c2cnc3ccccc3c2)C[C@@H](C)C(=O)[C@H](C)[C@H]2NC(=O)O[C@@]21C]
(1R,2R,4R,6S,7R,8R,10R,13R,14S)-7-[(2S,3R,4S,6R)-4-(dimethylamino)-3-hydroxy-6-methyloxan-2-yl]oxy-13-ethyl-2,4,6,8,10,14-hexamethyl-6-[(E)-3-quinolin-3-ylprop-2-enoxy]-12,15-dioxa-17-azabicyclo[12.3.0]heptadecane-3,9,11,16-tetrone (Cethromycin);
or a salt, solvate, stereoisomer, or prodrug thereof, and a pharmaceutically acceptable carrier.

2. The method of claim 1, wherein the compound is cethromycin.

3. The method of claim 1, wherein the Plasmodium infection is via infection from Plasmodium falciparum, Plasmodium berghei, and Plasmodium vivax.

4. (canceled)

5. The method of claim 1, wherein the pharmaceutical composition further comprises at least one or more other antimalarial compounds.

6. The method of claim 5, wherein the other antimalarial compound is selected from the group consisting of primaquine, artemisinins, sulfadoxine, pyrimethamine, doxycycline, azithromycin, atovaquone, tetracycline, antifolates including trimethoprim sulfamethoxazole, quinolones, and clindamycin.

7.-8. (canceled)

9. The method of claim 1, wherein the effective amount of the composition is in a concentration of between 0.1 mg/kg to 100 mg/kg.

10. The method of claim 9, wherein the compound in the pharmaceutical composition is cethromycin.
